(19) United States
(12) Patent Application Publication
(10) Pub. No.: US 2018/0014793 A1
(43) Pub. Date: Jan. 18, 2018

(54) System and Method for Health Monitoring

(71) Applicant: Audrey Sharon Bejo-Huerto, Carson, CA (US)

(72) Inventor: Audrey Sharon Bejo-Huerto, Carson, CA (US)

(21) Appl. No.: 15/210,691

(22) Filed: Jul. 14, 2016

(51) Int. Cl. A61B 5/00; A61B 5/0205; A61B 5/024; A61B 5/1455

(52) U.S. Cl. CPC A61B 5/746; A61B 5/7282; A61B 5/0205; A61B 5/6804; A61B 5/0002; A61B 5/7425; A61B 5/7405; A61B 5/02438; A61B 5/14552; A61B 2503/04; A61B 2560/0418

(57) ABSTRACT

The present disclosure provides a health monitoring apparatus for an infant. The apparatus includes a wearable monitoring device that includes a sensor in order to collect vital statistics of an infant. The wearable monitoring device is further connected to a mobile device through a network. The vital statistics collected by the wearable monitoring device are transmitted to the monitoring device. If the vital statistics received are above or below a threshold level for the corresponding vital statistics, the monitoring device generates a visual alarm displayed on the monitoring device and an audible alarm sounded through speaker system of the wearable monitoring device.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### FIELD

The present present disclosure relates to a health monitoring apparatus or more particularly; the present present disclosure relates to a wearable health monitoring apparatus to monitor vital statistics of an infant and alert parents if any of the vital statistics falls or rises from a threshold value.

### BACKGROUND

Infant's health is of utmost concern to the parents. Most of the parents, in these days, are working. This means that infants are generally tended by nannies or caregivers who can be old grandparents. Although, nannies or caregivers can take care of normal day to day health needs of babies, however, they might miss vital signs and symptoms concerning the babies' well-being. Sudden infant death syndrome is a serious problem that can occur. Furthermore, infants may be suffering from other serious conditions like congenital heart disease or respiratory problems which may not be so apparent to parents or nannies/caregivers.

Moreover, frequent illness of infants can result to irreversible damage to their health and will prove costly to the parents and the entire healthcare system in general. Parents or nannies may or may not be able to provide correct information to pediatrician about vital signs and symptoms. This can prove fatal to infant's health.

Therefore, in order to overcome the above mentioned shortcomings, there is a need for health monitoring devices.

### SUMMARY

This Summary is provided to introduce a selection of concepts in a simplified form that are further described below in the Detailed Description. This Summary is not intended to identify key features or essential features of the claimed subject matter, nor it is intended to be used to limit the scope of the claimed subject matter.

Various embodiments of the present disclosure solve the problems described above by providing a health monitoring apparatus that includes a wearable device. The wearable device obtains multiple vital statistics from body of a first user. The apparatus, further contains a display device that is operably connected to the wearable device. The display device receives the multiple vital statistics collected by the wearable device. These vital statistics are displayed to a second user and even store these as a history log. The display device is also configured to generate an alarm that may be visual or audio, in case any of the multiple vital statistics are on a threshold or crosses the same.

In another embodiment of the present disclosure there is provided a wearable monitoring device. The wearable monitoring device includes a sensor that obtains a plurality vital statistics of a user. The wearable monitoring device further contains a filter that filters different vital statistics of interest from the obtained plurality of vital statistics. It further contains a transmitter in order to transmit the filtered vital statistics of interest to a display device that may be remotely placed. It further has an alarm system that is further configured to receive instructions from the display device in an event if the level of vital statistics crosses a threshold level. The instructions are necessary for generating an alarm to alert about health of the user wearing the monitoring device.

In yet another embodiment of the present disclosure there is provided a computer implemented method for monitoring health of a user. The method includes receiving a plurality of vital statistics from a body worn sensor device. These vital statistics are then filtered and matched with the stored values of normal vital statistics. The method further includes tagging those vital statistics that cross predetermined threshold values with an abnormality tag and based on this tagging an alarm is sounded if any of the vital statistics cross their respective threshold value.

Additional features and advantages are realized through the techniques of the present disclosure. Other embodiments and aspects of the present disclosure are described in detail herein and are considered a part of the present disclosure. For a better understanding of the present disclosure with advantages and features, refer to the description and to the drawings.

### BRIEF DESCRIPTION OF THE DRAWINGS

The foregoing summary, as well as the following Detailed Description of preferred embodiments, is better understood when read in conjunction with the appended drawings. For the purposes of illustration, there is shown in the drawings exemplary embodiments; however, the present disclosure is not limited to the specific methods and instrumentalities disclosed. In the drawings:

FIG. 1A illustrates front view of a wearable monitoring device according to an embodiment of the present disclosure.

FIG. 1B illustrates hind view of a wearable monitoring device according to another embodiment of the present disclosure

FIG. 2 illustrates a health monitoring apparatus according to an embodiment of the present disclosure.

FIG. 3 illustrates a method according to an embodiment of the present disclosure.

### DETAILED DESCRIPTION

The following description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Reference throughout this specification to “a select embodiment,” “one embodiment,” or “an embodiment” means that a particular feature, structure, or characteristic described in connection with the embodiment is included in at least one embodiment of the disclosed subject matter. Thus, appearances of the phrases “a select embodiment,” “in one embodiment,” or “in an embodiment” in various places throughout this specification are not necessarily referring to the same embodiment.

As used herein, the term “computing device” should be understood to refer to, without limitation, any combination of hardware, software, firmware and/or any other suitable components now known or later to be developed in the art, where such combination is capable of, without limitation processing data (e.g. converting or analyzing the content of such data) and/or controlling electronic components of a larger system.

Embodiments of the present disclosure are useful for all types of patients however, one important use of the present disclosure is in case of monitoring health of infants. Hence, the foregoing explanation and description will be described in relation to infants. However, that does not limit the scope of the present disclosure to systems and methods for health monitoring in infants alone

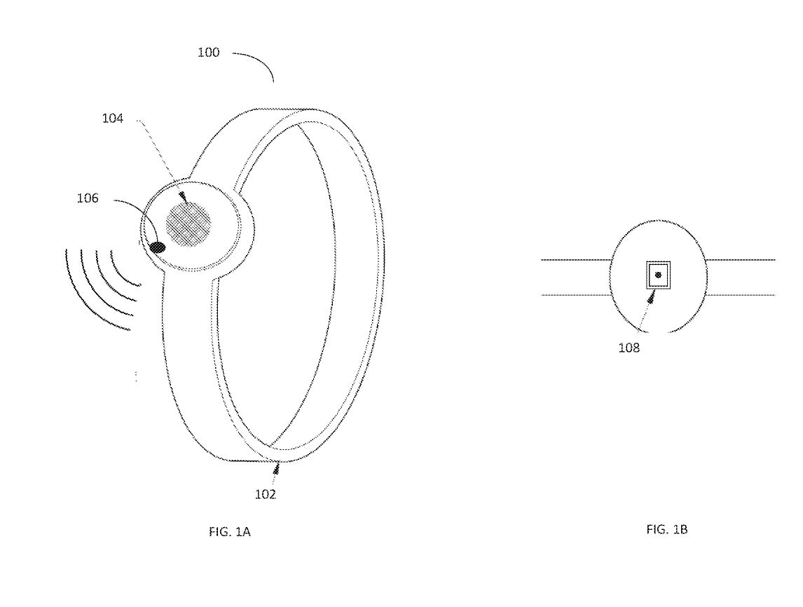

Referring to FIG. 1A through FIG. 1B which illustrates a wearable monitoring device 100 from front and back according to an embodiment of the present disclosure. In an embodiment of the present disclosure the wearable monitoring device 100 may be any other wearable device like ring, a garment etc. too. However, for explanation purpose, the wearable monitoring device has been depicted as a wrist band as shown in the FIG. 1a. The wearable monitoring device 100 includes a strap 102 that helps to tie the wearable monitoring device 100 to an infant's wrist. The strap 102 is hypoallergenic that is no allergy can be formed by using the strap 102. Also, the strap 102 is adjustable so as to be adjusted as per the growth of the infant.

Referring to FIG. 1b, the wearable monitoring device 100 includes a sensor 108 placed on the back side in order to obtain vital statistics of the infant. The vital statistics can be either heartbeat, or oxygen values or both. The oxygen values can be obtained by utilizing technology of a pulse oximeter.

Still referring to FIG. 1A, the wearable monitoring device 100. Also contains a speaker 104. The speaker 104 is utilized to sound an alarm that will be discussed later. The wearable monitoring device 100 further includes a transmitter 106. The transmitter 106 transmits the collected vital statistics to a remote device (not shown in Figure). The wearable monitoring device 100 is preferable battery powered that also powers the sensor 108. Such batteries may be a flexible thin film rechargeable battery. Flexible thin film batteries are ideally suited for the sensor 108 due to the fact that the strap 102 will be removably engaged around the infant's wrist. The thin film batteries have solid lithium cores rather than liquid cores, so they are less vulnerable to overheating and catching fire. They also lose virtually no power over time, and can be recharged thousands of times before they need to be replaced. While flexible thin film batteries have been shown and described, it will be appreciated by those skilled in the art that other batteries and battery technologies now known or later developed may be substituted without departing from the spirit and scope of the present disclosure.

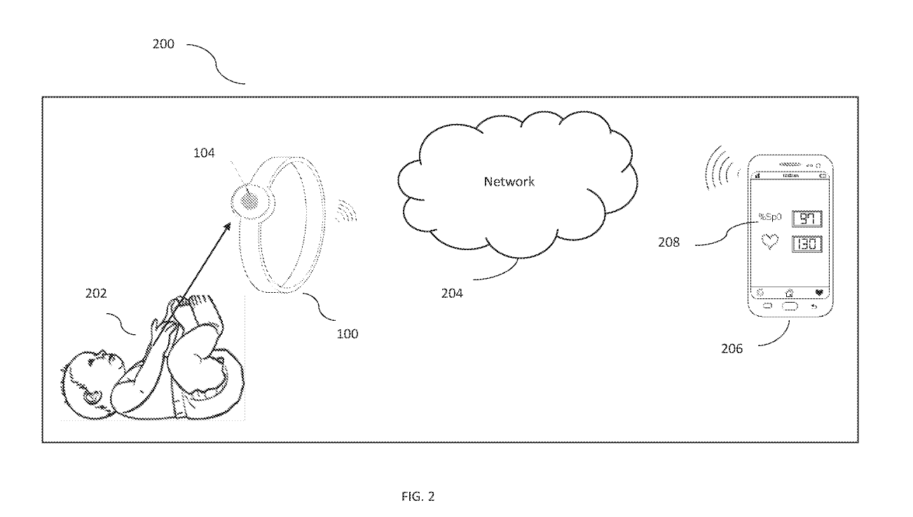

Now referring to FIG. 2 that illustrates a health monitoring apparatus 200. The health monitoring apparatus 200 is utilized to keep in check health of an infant 202. The health monitoring apparatus 200, includes a wearable device 100 in order to collect vital statistics of the infant 202. The wearable monitoring device 100 is placed on the infant's 202 wrist. The wearable monitoring device 100 as described above includes a sensor 108 that collects the various vital statistics of the infant 202. These vital statistics can be either heartbeat, oxygen values or both of them. These collected values are then transmitted to a remote display device 206 through a network 204. The network 204 can be either a wired or wireless network. The wireless network may be a short range wireless device like a ZigBee protocol, Wi-Fi, Bluetooth, near field communication protocol, or any other any suitable protocol now known or later developed. Also, the long range wireless network may be utilized like mobile network, cloud, world wide web, 3G, 4G, etc.

In an embodiment of the present disclosure, the display device could be a mobile phone, a laptop, a tablet computer or a personal digital assistant. The vital statistics can be displayed on a user interface 208 of the display device 206. The user interface 208 could be a part of an application developed specially for monitoring infant's health. The application can be developed using any of the programming languages like JAVA, C++, C#, C, etc. for any operating system like android, iOS, windows etc. The application also uses internal memory of the display device 206 in order to store normal values and threshold values for each of vital statistics being collected by the wearable monitoring device 100. When the display device 206 receives the vital statistics' values from the wearable monitoring device 100, it matches the values of the collected vital statistics with the pre-stored values and thresholds. These values are also displayed to user of the display device 206 that may be infant's parents or doctor. Also, to keep a medical history, these values are stored within the internal memory of the display device 206. For babies with medical condition (heart or respiratory), this can be very useful.

The data can be studied by infant's pediatrician. The health monitoring apparatus 200 will not only measure the baby's heart rate, and oxygen saturation but can also provide useful information for the parents and physicians. Since the data collected is also stored and this stored data can provide important information to the pediatrician. For e.g. how often did the infant have low heart rate or low oxygenation level. Such information can be difficult to remember or keep track of. Now, If the heart rate is below 100 or if the oxygen is below 90%, the health monitoring apparatus 100 will alarm providing a visual and audible signal. The audible alarm can be sounded either through the wireless monitoring device 100's speaker 104 or through a speaker system (not shown in FIG) of the display device 206. The alarm can also be a visual through the display 208 of the display device 206.

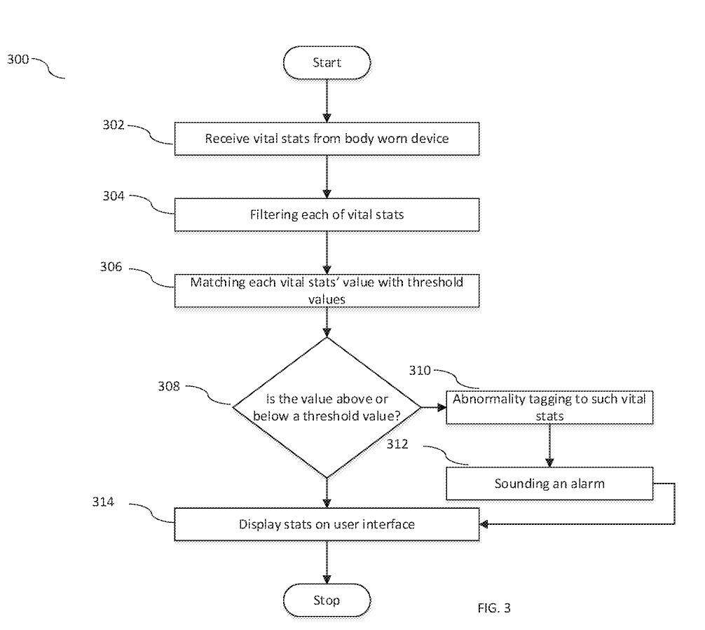

Now referring to FIG. 3 that illustrates a method 300 of monitoring health of an infant. The method 300 starts with step 302. At step 302, vital statistics values are received by a monitoring device from a body worn device. In an embodiment of the present disclosure, the vital statistics may be heartbeat, or oxygen values or both the values. In other embodiment of the present disclosure, the body worn device may be a wrist band, or a pen, or a garment. However, for ease of description, body worn device will be referred to be as a wrist band that may be placed on the infant's wrist whose health is to be monitored. In other embodiments of the present disclosure, the monitoring device could be a locally or remotely placed device. This device may be either a laptop, or a mobile phone or a tablet computer. For ease of description, this monitoring device will be referred to as a mobile phone that receives the vital statistics from the wrist band through a dedicated application.

Still referring to FIG. 3, the wrist band includes a sensor system that receives the vital statistics of the infant. The wrist band and the mobile phone are interconnected via a wireless network. Wireless network may be a Wi-Fi network, Bluetooth network, mobile network etc.

At step 304, the vital statistics received from the wrist band by the mobile phone are segregated into various vital statistics being monitored. Since the vital statistics received have mixed values therefore, this filtering is necessary to monitor different types of vital statistics. Further, at step 306, each of these vital statistics values are matched with predetermined and pre-stored normal values for each vital statistics and their corresponding threshold level values.

Furthermore, at step 308, determination is done as to whether the values of the received values have crossed the respective threshold values for corresponding vital statistics. If the threshold values are crossed then, an abnormality tag is attached to those vital statistics at step 310. Then, at 312, an alarm is sounded through the monitoring device as a visual alarm or an audible alarm through speaker placed on the wrist band. Also, further at step 314, these values are displayed on the monitoring device for ease of user i.e. parent using it.

Embodiments of the present disclosure may be embodied in other specific forms without departing from the spirit of essential attributes of the present disclosure.

While there has been shown and described herein what are presently considered the preferred embodiments of the present disclosure, it will be apparent to those skilled in the art that various changes and modifications can be made therein without departing from the scope of the present disclosure as defined by the appended claims.

While certain embodiments have been described, these embodiments have been presented by way of example only, and are not intended to limit the scope of the present disclosure. Indeed, the novel methods, devices, and systems described herein may be embodied in a variety of other forms. Furthermore, various omissions, substitutions, and changes in the form of the methods, devices, and systems described herein may be made without departing from the spirit of the present disclosure. The accompanying claims and their equivalents are intended to cover such forms or modifications as would fall within the scope and spirit of the present disclosure.

1. A health monitoring apparatus comprising:
a wearable device, wherein the device is configured to obtain a plurality of vital statistics of a first user;
a display device operably connected with the wearable monitoring device, wherein the display device is configured to;
receive the plurality of vital statistics from the wearable monitoring device;
display the vital statistics to a second user;
generate an alarm in case anyone of the plurality of vital statistics are on a threshold level; and
store the plurality of vital statistics to be analyzed later.

2. The health monitoring apparatus of claim 1, wherein the wearable monitoring device is anyone of a wrist band, a pen, a garment, or a ring.

3. The health monitoring apparatus of claim 1, wherein the first user is an infant.

4. The health monitoring apparatus of claim 1, wherein the second user is a parent.

5. The health monitoring apparatus of claim 1, wherein the display device is any one of a mobile device, a laptop, a tablet computer, a smart watch, or a personal digital assistant.

6. The health monitoring apparatus of claim 1, wherein plurality the vital statistics is any one or a combination of a heartbeat, or oxygen level.

7. The health monitoring apparatus of claim 1, wherein the wearable monitoring device and the display device are connected through a wireless network.

8. The health monitoring apparatus of claim 1, wherein the alarm is one of: a visual alarm and an audio alarm.

9. The health monitoring apparatus of claim 8, wherein the visual alarm is on the display device.

10. The health monitoring apparatus of claim 8, wherein the display device and the wearable monitoring device include a speaker system.

11. The health monitoring apparatus of claim 10, wherein the speaker system is configured for the audio alarm.

12. A wearable monitoring device comprising:
a sensor mounted on the wearable monitoring device configured to obtain a plurality of vital statistics of a user;
a filter configured to filter different vital statistics of interest from the obtained plurality of vital statistics;
a transmitter configured to transmit the vital statistics of interest to a display device; and an alarm system,
wherein the alarm system is configured to;
receive instructions from the display device if the level of the vital statistics of interest falls or rises above a threshold level; and
generate an alarm based on the instructions.

13. The wearable monitoring device of claim 12, wherein the sensor includes a transducer responsive to the plurality of vital statistics.

14. The wearable monitoring device of claim 13, wherein the user is an infant.

15. The wearable monitoring device of claim 13, wherein the plurality of vital statistics is any one or a combination of a heartbeat, or oxygen level.

16. The wearable monitoring device of claim 13, wherein the wearable monitoring device and the display device are connected via a wireless network.

17. The wearable monitoring device of claim 16, wherein the alarm is an audio alarm.

18. A computer implemented method comprising:
receiving, by a computing device, a plurality of vital statistics from a body worn sensor device;
filtering and matching, by the computing device, values of the plurality of vital statistics with stored values of normal vital statistics;
tagging, by the computing device, an abnormality tag to vital statistics if the value for them falls or rises above a threshold value; and
sounding an alarm, by the computing device, based on the values of the vital statistics crossing the threshold value.

19. The computer implemented method of claim 18, wherein the method further comprises causing, by the computing device, the vital statistics to be displayed on a screen of a display device.

20. The computer implemented method of claim 19, wherein the display device is a mobile device.
